(19) United States
(12) Patent Application Publication
(10) Pub. No.: US 2002/0119113 A1
(43) Pub. Date: Aug. 29, 2002

(54) SHAMPOO COMPOSITIONS

(76) Inventors: FRANCES Ann ELLIS, WIRRAL (GB); JONATHAN David HAGUE, JAKARTA (GB); MATTHEW Leslie PEARCE, WIRRAL (GB)

Correspondence Address: UNILEVER, PATENT DEPARTMENT, 45 RIVER ROAD, EDGEWATER, NJ, 07020, US

(21) Appl. No.: 09/433,162

(22) Filed: Nov. 3, 1999

(30) Foreign Application Priority Data
Nov. 3, 1998 (GB) 9824024.5

(51) Int. Cl. A61K 7/06; A61K 7/075; A61K 7/08

(52) U.S. Cl. 424/70.22; 424/70.1

(57) ABSTRACT

A shampoo composition comprising an active ingredient and multilamellar vesicles, characterised in that the multilamellar vesicles consist essentially of anionic surfactant and a sterol.

### FIELD OF THE INVENTION

This invention relates to shampoo compositions, more particularly to shampoo compositions which incorporate multilamellar vesicles, which compositions provide improved encapsulation and delivery of active ingredients incorporated therein.

### BACKGROUND OF THE INVENTION AND PRIOR ART

When treating hair with rinse-off products incorporating active ingredients, a considerable amount of the active ingredient will be rinsed away, especially if the active ingredient is surfactant-soluble. Studies have shown that the level of retention in the case of a simple shampoo composition, where the active ingredient is solubilised in the surfactant micelles in the product, can be as low as 5%.

A further problem associated with the incorporation of active ingredients into hair treatment compositions is that they may be sensitive to such factors such as heat, oxidation, light, moisture, pH and (particularly in the case of nutrient active ingredients such as amino acids) microbial attack. They may also tend to react with other ingredients in a formulation.

It would be desirable to provide a shampoo composition with improved encapsulation and delivery of active ingredients incorporated therein, as this would provide better performance of the active ingredient and the option of reducing the level of expensive active ingredient in the product, with consequent cost saving.

The manufacture of liposomes, i.e. lipid vesicles, and their use for the encapsulation and delivery of a wide variety of materials is well known (see, for example New, R.R.C., ed. Liposomes—a practical approach, Oxford University Press). Most of the commonly used liposomes are single-layered vesicles prepared from phospholipids. The reason for this is that phospholipids are the principal structural components of natural membranes. However, phospholipids are susceptible to enzymatic degradation, autoxidation, and acidic pH conditions, and are exceedingly expensive to prepare. Furthermore, phospholipid vesicles are relatively fragile, tending to rupture, coalesce, and release their encapsulates.

To avoid these disadvantages, there has been a movement towards the development of vesicles prepared from a variety of lipids and surfactants. For example, WO-A-9 219 214 describes oil-in-water emulsion compositions for artificial tanning of human skin containing lipid vesicles formed from a polyoxyalkylene alkyl ether, a sterol, a quaternary ammonium compound, and optionally a non-surface active oil. WO95/23578 describes a hair conditioner in which the multilamellar vesicles are formed by blending cationic surfactant and cholesterol.

### SUMMARY OF THE INVENTION

The inventors have surprisingly found that multilamellar vesicles are spontaneously formed when a shampoo base comprising anionic surfactant is added to a dispersion of cholesterol. The multilamellar vesicles are storage-stable and can be used to encapsulate active ingredients in the shampoo, thereby enhancing the performance and delivery of the active ingredients from the shampoo.

Accordingly, the present invention provides a shampoo composition comprising an active ingredient and multilamellar vesicles, characterised in that the multilamellar vesicles consist essentially of anionic surfactant and a sterol.

### DETAILED DESCRIPTION OF THE INVENTION

The inventors have found that the addition of an aqueous dispersion of a sterol to a shampoo base comprising at least one anionic surfactant induces the formation of multilamellar vesicles. The structures so generated may be easily visualised using conventional contrast microscopy techniques.

The aqueous dispersion of a sterol (suitably cholesterol) may suitably be prepared by adding solid sterol (suitably solid cholesterol) to water, heating, mixing at high shear, and leaving the mixture to cool to room temperature while stirring.

A specific example of a suitable process for preparing such an aqueous dispersion may be described as follows:

(a) Heat water up to 60-80° C.;

(b) Add solid cholesterol with constant stirring using a magnetic stirrer for 1 hour;

(c) Mix with high shear (a suitable instrument is an Ystral mixer, 5 mm probe, setting 7) for 30 seconds;

(d) Place back on magnetic stirrer and stir until cool, by which time the mixture is a uniform dispersion.

Multilamellar vesicles formed according to the invention are storage-stable and can be used to encapsulate active ingredients in the shampoo, thereby enhancing the performance and delivery of the active ingredients from the shampoo. Microscopy studies of shampoo compositions prepared according to the invention demonstrate at least partial encapsulation of such incorporated active ingredient in the multilamellar vesicles. It is preferable to optimise such encapsulation to allow the best efficiency of deposition enhancement from the shampoo compositions of the invention, particularly where soluble or solubilised active ingredients are concerned. In general, it has been found that optimal encapsulation of active ingredient(s) occurs when the active ingredient(s) for encapsulation are added prior to the formation of the multilamellar vesicles. This process enables optimal incorporation of the active ingredient(s) when the multilamellar vesicles are formed.

Accordingly, a preferred process for preparation of a shampoo composition according to the invention comprises the following steps:

(i) forming an aqueous dispersion of a sterol;

(ii) adding an active ingredient to the aqueous dispersion so obtained, and

(iii) adding to the mixture obtained in (ii) a shampoo base comprising at least one anionic surfactant.

The ingredients of the shampoo compositions of the invention may be characterised in more detail as follows:

Sterol

A sterol is an essential component of the shampoo compositions of this invention. Sterols are described in Hackh's Chemical Dictionary 4th ed., p.638 (McGraw-Hill, New York; 1972). A preferred sterol for use in the present invention is cholesterol.

The level of sterol, preferably cholesterol, in the shampoo composition is suitably from 0.005% to 2%, preferably 0.00625% to 1%, optimally 0.025% to 0.05% by weight based on the total weight of the shampoo composition.

Active Ingredient

Among suitable active ingredients are:

(i) sunscreens. Among suitable sunscreens are, camphor derivatives, benzophenone compounds such as 4,4′-tetrahydroxy-benzophenone, sold commercially as Uvinul D50, and 2-hydroxy-4-methoxybenzophenone, sold commercially as Eusolex 4360, dibenzoyl methane derivatives such as t-butyl-4-methoxydibenzoylmethane, sold commercially as Parsol 1789, and isopropyldibenzoyl methane, sold commercially as Eusolex 8020. Preferred sunscreen materials are cinnamates, such as 2-ethylhexyl-p-methoxy cinnamate, sold commercially as Parsol MCX, 2-ethoxy ethyl-p-methoxy cinnamate, sold commercially as Giv-Tan F and isoamyl-p-methoxy cinnamate, sold commercially as Neo-Heliopan E1000.

(ii) antidandruff agents, such as zinc pyrithione, and other 1-hydroxy pyridones. A preferred antidandruff agent is the 1-hydroxy-2-pyridone derivative known as piroctone olamine, whose chemical name is 1-hydroxy-4-methyl-6-(2,4,4-trimethylpentyl)-2-pyridone monoethanolamine salt, and which is sold under the trade name OCTIPIROX by Hoechst AG.

(iii) natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts. A particularly preferred amino acid nutrient is arginine.

(iv) hair fibre benefit agents. Examples are:

ceramides, for moisturising the fibre and maintaining cuticle integrity. Ceramides are available by extraction from natural sources, or as synthetic ceramides and pseudoceramides. A preferred ceramide is Ceramide II, ex Quest. Mixtures of ceramides may also be suitable, such as Ceramides LS, ex Laboratoires Serobiologiques.

fatty acids, for cuticle repair and damage prevention. Examples are branched chain fatty acids such as 18-methyleicosanoic acid and other homologues of this series, straight chain fatty acids such as stearic, myristic and palmitic acids, and unsaturated fatty acids such as oleic acid, linoleic acid, linolenic acid and arachidonic acid. A preferred fatty acid is oleic acid. The fatty acids may be added singly, as mixtures, or in the form of blends derived from extracts of, e.g. lanolin.

Mixtures of any of the above active ingredients may also be used. A particularly preferred combination is arginine and oleic acid.

The active ingredient is normally present in a concentration of from 0.005% to 5%, preferably from 0.085% to 2% by total weight of active ingredient based on the total weight of the shampoo composition.

Anionic Surfactant

Suitable anionic surfactants for shampoo compositions of the invention include the alkyl sulphates, alkyl ether sulphates, alkaryl sulphonates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates and acyl methyl taurates, especially their sodium, magnesium ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from one to 10 ethylene oxide or propylene oxide units per molecule, and preferably contain 2 to 3 ethylene oxide units per molecule.

Examples of suitable anionic surfactants include sodium lauryl sulphate, sodium lauryl ether sulphate, ammonium lauryl sulphosuccinate, ammonium lauryl sulphate, ammonium lauryl ether sulphate, sodium dodecylbenzene sulphonate, triethanolamine dodecylbenzene sulphonate, sodium cocoyl isethionate, sodium lauroyl isethionate, sodium N-lauryl sarcosinate, and mixtures thereof.

The level of anionic surfactant in shampoo compositions of the invention is generally from 3 to 50%, preferably from 3 to 30%, more preferably from 5% to 20% by weight of the total composition.

Optional Ingredients

Additional Surfactant

The shampoo composition of the invention may also include co-surfactants, to help impart aesthetic, physical or cleansing properties to the composition. Preferred examples include amphoteric, zwitterionic and/or nonionic surfactants, which can be included in an amount ranging from 0% to about 10% by weight based on the total weight of the shampoo composition.

Examples of amphoteric or zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine, sodium cocamphopropionate, and mixtures thereof.

Representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Other representative nonionics include mono- or di-alkyl alkanolamides. Examples include coco mono- or di-ethanolamide and coco mono-isopropanolamide.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO-(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

Mixtures of any of the above nonionic surfactants may also be used.

Further surfactant may also be present as emulsifier for emulsified components of the shampoo composition, e.g. emulsified particles of silicone. This may be the same surfactant as the anionic surfactant or any co-surfactant, or may be different.

Suitable emulsifying surfactants are well known in the art and include anionic and nonionic surfactants. Examples of anionic surfactants used as emulsifiers for materials such as silicone particles are alkylarylsulphonates, e.g., sodium dodecylbenzene sulphonate, alkyl sulphates e.g., sodium lauryl sulphate, alkyl ether sulphates, e.g., sodium lauryl ether sulphate nEO, where n is from 1 to 20 alkylphenol ether sulphates, e.g., octylphenol ether sulphate nEO where n is from 1 to 20, and sulphosuccinates, e.g., sodium dioctylsulphosuccinate.

Examples of nonionic surfactants used as emulsifiers for materials such as silicone particles are alkylphenol ethoxylates, e.g., nonylphenol ethoxylate nEO, where n is from 1 to 50, alcohol ethoxylates, e.g., lauryl alcohol nEO, where n is from 1 to 50, ester ethoxylates, e.g., polyoxyethylene monostearate where the number of oxyethylene units is from 1 to 30.

The total amount of surfactant (including any co-surfactant, and/or any emulsifier) in shampoo compositions of the invention is generally from 0.1 to 50% by weight, preferably from 5 to 30%, more preferably from 10% to 25% by weight of the total shampoo composition.

Conditioning Agents

The shampoo composition of the invention may also include one or more conditioning agents. As used herein, the term “conditioning agent” includes any material which is used to give a particular conditioning benefit to hair and/or skin. For example, in shampoo compositions for use in washing hair, suitable materials are those which deliver one or more benefits relating to shine, softness, combability, wet-handling, anti-static properties, protection against damage, body, volume, stylability and manageability.

Preferred conditioning agents for use in the present invention include emulsified silicones, used to impart for example wet and dry conditioning benefits to hair such as softness, smooth feel and ease of combability.

The silicone is insoluble in the aqueous matrix of the composition and so is present in an emulsified form, with the silicone present as dispersed particles.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums. “Silicone gum” denotes polydiorganosiloxanes having a molecular weight of from 200,000 to 1,000,000 and specific examples include dimethicone gums, dimethiconol gums, polydimethyl siloxane/diphenyl/methylvinylsiloxane copolymers, polydimethylsiloxane/methylvinylsiloxane copolymers and mixtures thereof. Examples include those materials having a slight degree of cross-linking, as are described for example in WO 96/31188. These materials can impart body, volume and stylability to hair, as well as good wet and dry conditioning.

The viscosity of the emulsified silicone itself (not the emulsion or the final hair conditioning composition) is typically at least 10,000 cst. In general we have found that conditioning performance increases with increased viscosity. Accordingly, the viscosity of the silicone itself is preferably at least 60,000 cst, most preferably at least 500,000 cst, ideally at least 1,000,000 cst. Preferably the viscosity does not exceed 109 cst for ease of formulation.

Emulsified silicones for use in hair shampoos and conditioners of the invention will typically have an average silicone particle size in the composition of less than 30, preferably less than 20, more preferably less than 10 microns. We have found that reducing the particle size generally improves conditioning performance. Most preferably the average silicone particle size of the emulsified silicone in the composition is less than 2 microns, ideally it ranges from 0.01 to 1 micron. Silicone emulsions having an average silicone particle size of <0.15 microns are generally termed microemulsions.

Particle size may be measured by means of a laser light scattering technique, using a 2600D Particle Sizer from Malvern Instruments.

Suitable silicone emulsions for use in the invention are also commercially available in a pre-emulsified form.

Examples of suitable pre-formed emulsions include emulsions DC2-1766, DC2-1784, and microemulsions DC2-1865 and DC2-1870, all available from Dow Corning. These are all emulsions/microemulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum. A further preferred example is the material available from Dow Corning as DC X2-1391, which is a microemulsion of cross-linked dimethiconol gum.

A further preferred class of silicones for inclusion in shampoos and conditioners of the invention are amino functional silicones. By “amino functional silicone” is meant a silicone containing at least one primary, secondary or tertiary amine group, or a quaternary ammonium group.

Pre-formed emulsions of amino functional silicones are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2-8154 (all ex Dow Corning).

The amount of silicone incorporated into the compositions of the invention depends on the level of conditioning desired and the material used. A preferred amount is from 0.01 to about 10% by weight of the total composition although these limits are not absolute. The lower limit is determined by the minimum level to achieve conditioning and the upper limit by the maximum level to avoid making the hair and/or skin unacceptably greasy. We have found that an amount of silicone of from 0.5 to 1.5% by weight of the total composition, is a particularly suitable level.

A further preferred class of conditioning agents are peralk(en)yl hydrocarbon materials, used to enhance the body, volume and stylability of hair.

EP 567 326 and EP 498 119 describe suitable peralk(en)yl hydrocarbon materials for imparting stylability and enhanced body to hair. Preferred materials are polyisobutylene materials available from Presperse, Inc. under the PERMETHYL trade name.

The amount of per-alk(en)yl hydrocarbon material incorporated into the compositions of the invention depends on the level of body and volume enhancement desired and the specific material used. A preferred amount is from 0.01 to about 10% by weight of the total composition although these limits are not absolute. The lower limit is determined by the minimum level to achieve the body and volume enhancing effect and the upper limit by the maximum level to avoid making the hair unacceptably stiff. We have found that an amount of per-alk(en)yl hydrocarbon material of from 0.5 to 2% by weight of the total composition is a particularly suitable level.

Cationic Deposition Polymer

A cationic deposition polymer is a preferred ingredient which may be included in shampoo compositions of the invention, for enhancing conditioning performance of the shampoo. By “deposition polymer” is meant an agent which enhances deposition of active ingredients and/or conditioning components (such as silicones) from the shampoo composition onto the intended site during use, i.e. the hair and/or the scalp.

The deposition polymer may be a homopolymer or be formed from two or more types of monomers. The molecular weight of the polymer will generally be between 5 000 and 10 000 000, typically at least 10 000 and preferably in the range 100 000 to about 2 000 000. The polymers will have cationic nitrogen containing groups such as quaternary ammonium or protonated amino groups, or a mixture thereof.

The cationic nitrogen-containing group will generally be present as a substituent on a fraction of the total monomer units of the deposition polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic deposition polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic deposition polymers can comprise mixtures of monomer units derived from amine- and/or quaternary ammonium-substituted monomer and/or compatible spacer monomers.

Suitable cationic deposition polymers include, for example:

copolymers of 1-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16. This material is commercially available from BASF Wyandotte Corp. (Parsippany, N.J., USA) under the LUVIQUAT tradename (e.g. LUVIQUAT FC 370);

copolymers of 1-vinyl-2-pyrrolidine and dimethylaminoethyl methacrylate, referred to in the industry (CTFA) as Polyquaternium-11. This material is available commercially from Gaf Corporation (Wayne, N.J., USA) under the C-AFQUAT tradename (e.g., GAFQUAT 755N);

cationic diallyl quaternary ammonium-containing polymers including, for example, dimethyldiallylammonium chloride homopolymer and copolymers of acrylamide and dimethyldiallylammonium chloride, referred to in the industry (CTFA) as Polyquaternium 6 and Polyquaternium 7, respectively;

mineral acid salts of amino-alkyl esters of homo-and co-polymers of unsaturated carboxylic acids having from 3 to 5 carbon atoms, (as described in U.S. Pat. No. 4,009,256);

cationic polyacrylamides(as described in WO95/22311).

Other cationic deposition polymers that can be used include cationic polysaccharide polymers, such as cationic cellulose derivatives, cationic starch derivatives, and cationic guar gum derivatives.

Cationic polysaccharide polymers suitable for use in compositions of the invention include those of the formula:

A—O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (Commercially available from Rhone-Poulenc in their JAGUAR trademark series).

Examples are JAGUAR C13S, which has a low degree of substitution of the cationic groups and high viscosity. JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Preferably the cationic deposition polymer is selected from cationic cellulose and cationic guar derivatives.

Particularly preferred deposition polymers are JAGUAR C13S, JAGUAR C15, JAGUAR C17 and JAGUAR C16 and JAGUAR C162.

The cationic deposition polymer will generally be present at levels of from 0.001 to 5%, preferably from about 0.01 to 1%, more preferably from about 0.02% to about 0.5% by weight of the total composition.

As further optional components for inclusion in the compositions of the invention, in addition to water, the following may be mentioned: pH adjusting agents, viscosity modifiers, pearlescers, opacifiers, suspending agents, preservatives, colouring agents, dyes, proteins, herb and plant extracts, polyols and other moisturising and/or conditioning agents.

Embodiments of the present invention will now be further illustrated by reference to the following examples. All amounts given are in % by weight based on total weight of the composition, unless otherwise stated.

### EXAMPLE

A shampoo composition was prepared having the following ingredients:

| Ingredient | wt % |
|---|---|
| Sodium lauryl ether sulphate (2EO) | 9 |
| Cocamidopropyl betaine | 5 |
| L-Arginine | 0.2 |
| Cholesterol | 0.05 |
| Oleic acid | 0.01 |
| Water, minors | to 100 |

The composition was prepared by dissolving the cholesterol in water at 60° C., and stirring overnight at room temperature. The oleic acid and arginine were then added, followed by the remaining ingredients, using an Ystral mixer to combine the ingredients and form an emulsion.

The composition was observed to contain multilamellar vesicles, as evidenced by centrifugation of a 1 ml aliquot at 13,000 rpm for 15 minutes, and examination of the resulting precipitate by polarised light microscopy at ×20 magnification. Clusters of multilamellar vesicles were evident under the microscope, distinguished by their “Maltese Cross” appearance.

The multilamellar vesicles are stable to centrifugation and recentrifugation. This therefore provides a convenient route to producing more concentrated dispersions of multilamellar vesicles for incorporation into hair formulations, if desired.

1. A shampoo composition comprising an active ingredient and multilamellar vesicles, characterised in that the multilamellar vesicles consist essentially of anionic surfactant and a sterol.

2. A shampoo composition according to claim 1, in which the sterol is cholesterol.

3. A shampoo composition according to claims 1 or 2, in which the active ingredient is selected from the group consisting of amino acids, fatty acids and mixtures thereof.

4. A shampoo composition according to any preceding claim, in which the anionic surfactant is selected from the group consisting of sodium lauryl sulphate, sodium lauryl ether sulphate, ammonium lauryl sulphosuccinate, ammonium lauryl sulphate, ammonium lauryl ether sulphate, sodium dodecylbenzene sulphonate, triethanolamine dodecylbenzene sulphonate, sodium cocoyl isethionate, sodium lauroyl isethionate, sodium N-lauryl sarcosinate, and mixtures thereof.

5. A shampoo composition according to any preceding claim, further comprising an amphoteric or zwitterionic surfactant selected from the group consisting of lauryl amine oxide, cocodimethyl sulphopropyl betaine and preferably lauryl betaine, cocamidopropyl betaine, sodium cocamphopropionate, and mixtures thereof.

6. A process for preparation of a shampoo composition comprising multilamellar vesicles and an active ingredient, the process comprising the following steps:
(i) forming an aqueous dispersion of a sterol;
(ii) adding an active ingredient to the aqueous dispersion so obtained, and
(iii) adding to the mixture obtained in (ii) a shampoo base comprising at least one anionic surfactant.
